(19) United States
(12) Patent Application Publication
(10) Pub. No.: US 2023/0249156 A1
(43) Pub. Date: Aug. 10, 2023

(54) ELECTROSPUN FIBROUS MEMBRANES AND USES THEREOF

(71) Applicant: TECHNION RESEARCH & DEVELOPMENT FOUNDATION LIMITED, Haifa (IL)

(72) Inventors: Eyal ZUSSMAN, Haifa (IL); Rita VILENSKY, Haifa (IL); Ophir MAROM, Haifa (IL)

(21) Appl. No.: 18/107,641

(22) Filed: Feb. 9, 2023

(30) Foreign Application Priority Data
Feb. 10, 2022 (IL) 290554

(51) Int. Cl. B01J 20/28; G01N 1/22; B01J 20/26; G01N 27/622; G01N 33/497; D04H 1/56; D04H 1/4366; D04H 1/728

(52) U.S. Cl. CPC B01J 20/28038; G01N 1/2247; B01J 20/262; B01J 20/28004; B01J 20/28011; B01J 20/28059; B01J 20/2808; B01J 20/28083; B01J 20/28085; G01N 27/622; G01N 33/497; D04H 1/56; D04H 1/4366; D04H 1/728; G01N 2033/4975; D10B 2401/10; D10B 2401/063; D10B 2505/00

(57) ABSTRACT

Fibrous mats composed of polymeric fibers comprising an aromatic polymer are provided. Further, articles and methods of use of the fibrous mats, including, but not limited to filters and membranes for sampling of fluid samples, are also provided.

### CROSS-REFERENCE

This application claims the benefit of priority to IL Patent Application No. 290554 filed Feb. 10, 2022 entitled “ELECTROSPUN FIBROUS MEMBRANES AND USES THEREOF”, the contents of which are incorporated herein by reference in their entirety.

### FIELD OF THE INVENTION

The present invention, in some embodiments thereof, relates to a fibrous mat composed of polymeric fibers comprising an aromatic polymer, preparation and use thereof.

### BACKGROUND OF THE INVENTION

Sampling of volatile organic compounds (VOC) in air is of high importance in a variety of disciplines such as: environmental sciences, biomarkers-sampling, food, flavor and fragrances, security and forensic sciences.

The conventional technique for air sampling and pre-concentration of VOCs is based on solid phase extraction (SPE), where the solute adsorbs onto solid sorbent. The adsorbed solutes are then desorbed by liquid extraction or thermal desorption and further transferred for analysis using various analytical equipment as ion mobility spectrometry (IMS), gas chromatography-mass spectroscopy (GC-MS) or thermal desorption mass spectrometry (TD-GC-MS).

Air sampling can be conducted via two modes: passive or dynamic. Passive air sampling involves the diffusive uptake of a chemical vapor by a sorbent over time. Dynamic air samplers rely on a pump to pass a volume of air through or past a sorbent.

Traditionally, sampling procedures have utilized SPE cartridges, where the adsorbent is packed into a column. Nevertheless, in the last decades SPE membranes has emerged as a promising alternative to a conventional cartridge. The SPE membranes possess several advantages over the conventional SPE cartridges, as lower pressure drop which allows considerably higher sampling flow rates, and direct integration with analyzing equipment including portable ion mobility spectrometry and thermal desorption mass spectrometry.

Tenax is a known trademark of a poly-2,6-diphenyl-p-phenylene oxide (PPPO), which is widely used as a sorbent material due to its exceptional features as high thermal stability, low water retention, and high oxygen resistance. Tenax is used as a column packing material for trapping volatiles from air (VOC) or liquids, and is recommended for retaining VOCs, for example, as suggested in ISO 16000-6. Nevertheless, the commercially available granular Tenax has significant limitations as low surface area which impedes its adsorption efficiency. Moreover, the conventional Tenax columns restrict its application when high sampling flow rates are required. Thus, a membrane-type adsorber, with enhanced surface area and adaptable geometry is advantageous in comparison to granular-type sorbent for VOC's sampling.

Accordingly, there is a need for novel sampling membranes having increased surface area and thus enhanced adsorption efficiency. Specifically, it is highly desirable to develop novel membranes capable of adsorbing sufficient amounts of VOCs to facilitate detection of specific analytes present at sub-ppb level within a fluid sample.

### SUMMARY OF THE INVENTION

In one aspect of the invention, there is provided a fibrous mat comprising a plurality of fibers, wherein each of the fibers is a porous polymeric fiber comprising an aromatic polymer; wherein: an average cross-section of the fibers is between 0.5 and 30 um; the aromatic polymer is stable at a temperature of at least 200° C.; the fibrous mat is characterized by air permeability coefficient of at least 10⁻¹³ m².

In one embodiment, the aromatic polymer is capable of binding a volatile organic specie (VOC).

In one embodiment, the aromatic polymer comprises poly-2,6-diphenyl-p-phenylene oxide.

In one embodiment, binding is by adsorption.

In one embodiment, the VOC is a non-polar molecule.

In one embodiment, the VOC comprises an alcohol, a glycol, a terpene, a cyclic compound, an aromatic compound, a chlorinated aromatic compound, a nitro-aromatic compound, an aldehyde, a ketone, an amine, or an amide, including any combination thereof.

In one embodiment, the fibrous mat is characterized by a density of between 0.05 and 0.1 gr/cm³.

In one embodiment, the fibrous mat is characterized by a porosity of between 70 and 90%.

In one embodiment, the fibrous mat is characterized by a BET surface area of at least 25 m²/g.

In one embodiment, the fibers are characterized by average pore size of between 1 and 500 nm.

In one embodiment, the fibrous mat is characterized by an average pore size of between 5 and 50 um.

In one embodiment, the fibrous mat is in a form of a self-supporting membrane, characterized by a thickness of between 50 and 2000 um.

In one embodiment, the self-supporting membrane is configured to support gas flow at a flow velocity of at most 5 m/sec.

In one embodiment, the self-supporting is in a form of a non-woven uniform layer.

In one embodiment, the self-supporting membrane is characterized by a tensile strength of at least 0.4 MPa.

In one embodiment, the self-supporting membrane is characterized by air permeability coefficient of between 10⁻¹² and 10⁻¹⁴ m².

In one embodiment, the self-supporting membrane is configured for sampling a VOC.

In one embodiment, the self-supporting membrane is capable of adsorbing the VOC at a w/w ratio between the VOC and the membrane between 1:1.000.000 and 1:10.

In one embodiment, the fibrous mat is characterized by elongation to break of between 5 and 10%.

In one embodiment, the fibrous mat is characterized by Young's modulus between 5 and 10 MPa.

In one embodiment, the fibrous mat is characterized by thermal stability at a temperature up to 500° C.

In one embodiment, the fibrous mat has a DSC pattern exhibiting at least one endothermic peak in the range of from 470° C. to 500° C.

In one embodiment, a Tg of the aromatic polymer within the fibrous mat is about 223° C.

In one embodiment, a crystallinity degree of the aromatic polymer within the fibrous mat is 20-50%.

In another aspect, there is provided a method for detecting a VOC of interest within a sample, the method comprising: contacting the fibrous mat of the invention with a fluid sample comprising a plurality of VOCs under appropriate conditions, thereby obtaining one or more VOCs adsorbed to the fibrous mat; exposing the one or more VOCs adsorbed to the fibrous mat to a detector configured for detecting the analyte of interest, thereby determining the presence of the VOC of interest within the sample.

In one embodiment, appropriate conditions comprise exposing the fibrous mat to a flowing gaseous sample at a flow rate of at least 0.05 m/sec.

In one embodiment, appropriate conditions comprise a time period sufficient for adsorbing an effective amount of the VOC of interest to the fibrous mat.

In one embodiment, the effective amount is sufficient for detection of the VOC of interest by the detector.

In one embodiment, a concentration of the VOC of interest within the sample is at least 0.1 ppb.

In another aspect, there is provided a method for manufacturing the fibrous mat of the invention, comprising: forming a polymeric solution, wherein the polymeric solution comprises the aromatic polymer and a solvent; and introducing the polymeric solution into an electrospinning apparatus under appropriate conditions, thereby obtaining the fibrous mat.

In one embodiment, the solvent is compatible with electrospinning process.

In one embodiment, the aromatic polymer has a sufficient solubility within the solvent.

In one embodiment, solubility comprises between 1 and 200 g/L.

In one embodiment, the solvent comprises a chlorinated solvent and a polar solvent.

In one embodiment, a v/v ratio between the chlorinated solvent and the polar solvent within the polymeric solution is between 7:3 and 9.5:0.5.

In one embodiment, the chlorinated solvent comprises chloroform, DCM, dichloroethane, or any combination thereof.

In one embodiment, the polar solvent comprises DMF, DMSO, NMP, methanol, ethanol, or any combination thereof.

In one embodiment, forming the polymeric solution comprises (i) dissolving a sufficient amount of the aromatic polymer within the chlorinated solvent to obtain a solution, and (ii) subsequently adding a sufficient amount of the polar solvent to the solution, thereby forming the polymeric solution.

In one embodiment, the method further comprises exposing the fibrous mat to a temperature between 100 and 300° C.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D are images presenting micrographs of High Resolution Scanning Electron Microscope (HRSEM) of the exemplary Tenax membranes of the invention at different magnifications electrospun from various polymeric solutions: chloroform:DMSO (84/16% v/v) abbreviated as ESM-A (1A1-1A3); chloroform:DMSO:EtOH (83%:7%:10%) abbreviated as ESM-B (1B1-1B3), chloroform/DMSO/EtOH (86%:7%:7%) abbreviated as ESM-C (1C); and chloroform/DMSO/EtOH (80%:7%:13%) abbreviated as ESM-D (1D).

FIG. 2 presents thermogravimetric analysis (TGA) thermographs of the exemplary Tenax electrospun membranes of the invention and of commercial Tenax beads.

FIG. 3 presents differential scanning coulometry (DSC) thermographs of “as-spun” exemplary Tenax electrospun membranes of the invention (ESM-A, ESM-B, ESM-C), and of commercial Tenax beads.

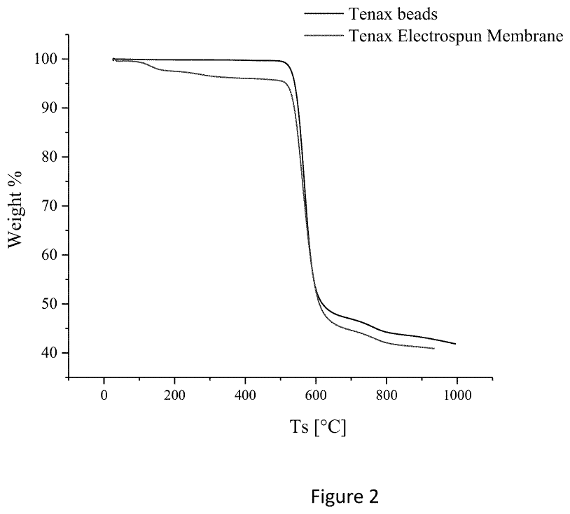

FIGS. 4A-4B are bar graphs presenting static adsorption of the tested VOC: MuskXylen (MX), naphthalene and thymol (4A) and of naphthalene (4B) on exemplary Tenax electrospun membranes of the invention (ESM-A, ESM-B, ESM-D) as compared to commercial column comprising Tenax beads (BEADS). Sampling time was about 3 hours.

FIG. 5 is a graph presenting a weight ratio between an adsorbed VOC (MuskXylen (MX)) and the Tenax-based sorbent including an exemplary Tenax electrospun membrane of the invention and a commercial column comprising Tenax beads. The VOC has been adsorbed to the Tenax-based sorbent by static adsorption.

FIG. 6A is a graph presenting the amount of Musk Xylene adsorbed on the Tenax membrane in comparison to Tenax column beads as a function of sampling flow rates, performed in a small-scale experimental set-up (analyte concentration 10 ppb).

FIGS. 6B-6C are images demonstrating: (6B) commercial TENAX column (Buchem. BV) and (6C) an exemplary Tenax membrane of the invention inside an adapter (membrane cross section, A=5 cm²).

FIG. 7 is a graph presenting the amount of Musk Xylene adsorbed on the Tenax membrane (A=5 cm²) vs. Tenax column as a function of sampling flow rates, performed in a large scale experimental set-up (analyte concentration 0.1 ppb).

FIG. 8A is a bar graph representing adsorption results obtained by interior air sampling of marine container in a field test experiment. Air sampling was performed via a dynamic sampling, at a flow velocity of 0.4 m/s. The bar graph presents the amount of Musk Xylene (at a concentration of about 0.1 ppb within the sampled air) adsorbed on an exemplary Tenax membrane of the invention (A=85 cm²) versus a commercial TENAX column.

FIG. 8B is an image presenting a custom-made gas sampler comprising a filter housing loaded with Tenax membrane used in the field experiment.

FIG. 9 is an image presenting an exemplary TENAX ESM of the invention, inserted into the inlet of thermal desorption unit of the portable Ion Mobility Spectrometer (MMTD—Smiths Detection).

FIG. 10 is a graph presenting a chromatogram of the breath sample as measured by a portable Ion Mobility Spectrometer (IMS).

### DETAILED DESCRIPTION OF THE INVENTION

In one aspect, there is provided a fibrous mat in a form of a fibrous matrix, wherein the fibrous matrix comprises polymeric fibers, wherein each of the polymeric fibers is a porous fiber comprising an aromatic polymer.

In some embodiments, the fibrous mat is in a form of one or more layers comprising a plurality of micron-sized polymeric fibers. In some embodiments, the fibrous mat comprises or is essentially composed of electrospun fibers. In some embodiments, the fibrous mat is in a solid state at a temperature up to 100° C., up to 200° C., up to 300° C., up to 400° C., or up to 470° C., including any range between.

As exemplified herein, the invention in some embodiments thereof, is based on the surprising finding that a Tenax (poly-2,6-diphenyl-p-phenylene oxide)-based membrane composed of the electrospun fibers characterized by a significantly greater surface area (as compared to Tenax beads) is capable of adsorbing and subsequently releasing one or more volatile organic specie (VOC) from a gaseous sample. The inventors further successfully implemented the membrane disclosed herein for sampling and subsequent detection of VOCs present within a gaseous sample at a concentration of 0.1 ppb.

As used herein, the term “matrix” refers to one or more porous layers of polymeric fibers randomly, and/or under certain order or control, distributed therewithin. In some embodiments, the terms “fibrous mat” and “matrix” are used herein interchangeably. Matrix may further include any materials incorporated within and/or interposed between the layers. In some embodiments, the matrix comprises randomly oriented polymeric fibers. In some embodiments, each polymeric fiber within the matrix is in contact with at least one additional polymeric fiber. In some embodiments, the polymeric fibers are randomly distributed within the matrix, so obtain a three-dimensional mesh structure comprising a void space between the fibers. In some embodiments, the polymeric fibers are randomly distributed within the matrix thus forming a plurality of pores (or void space).

In some embodiments, the terms “layer”, and “film” are used herein interchangeably, and refer to a material having a substantially uniform-thickness. In some embodiments, the term “layer” refers to a substantially homogeneous material characterized by a substantially the same chemical composition and/or substantially the same three-dimensional structure. In some embodiments, layer is characterized by a homogenous or uniform feature within the entire layer, wherein the feature is selected from spatial distribution of the polymeric fibers, fiber thickness, pore size, porosity, thickness, including any range between. In some embodiments, the term “entire layer” refers to at least 50%, at least 60%, at least 70%, at least 80%, at least 90%, at least 95%, at least 97% of the surface and/or volume of the layer, including any range between.

In some embodiments, the fibrous mat is in a form of a non-woven layer. In some embodiments, the fibrous mat is in a form of a layer characterized by a relatively uniform thickness, a uniform spatial distribution of the polymeric fibers, or both. In some embodiments, the polymeric fibers are electrospun fibers (e.g. electrospun microfibers). In some embodiments, the polymeric fibers have a cylindrical shape. In some embodiments, the polymeric fibers have a ribbon-like shape.

### Polymeric Fibers

In one aspect, there is provided a polymeric fiber (e.g. electrospun microfiber) comprising an aromatic polymer, wherein the aromatic polymer is thermally stable at a temperature of at least 200° C., at least 250° C., at least 300° C., at least 350° C., at least 400° C., at least 420° C., at least 450° C., at least 470° C., including any range between. In some embodiments, the aromatic polymer is thermally stable at a temperature of up to 500° C., up to 470° C., or up to 450° C., including any range between. In some embodiments, as used herein the term “thermally stable” refers to the ability of the polymer to retain at least 90%, at least 95%, at least 97%, at least 99%, at least 99.9% of its initial weight upon exposure thereof to a temperature disclosed herein. Thermal stability can be assessed by thermogravimetric analysis (TGA).

In some embodiments, the aromatic polymer is characterized by a sufficient binding affinity to a volatile organic compound (VOC), so as to result in an efficient binding of the VOC to the polymer. As used herein, the term “VOC” refers to one or more organic small molecules (usually having a molecular weight less than 1000 Da, or less than 500 Da) characterized by a vapor pressure of at least 10⁻¹⁰ atm at 20° C. In some embodiments, the efficient binding is so as to obtain an effective amount of the VOC stably bound to the polymer. In some embodiments, the VOC is a non-polar hydrophobic compound. In some embodiments, the VOC is characterized by a dipole moment between 0 and 0.5, between 0 and 0.4, between 0 and 0.1, between 0.1 and 0.5, between 0.4 and 0.5, including any range between.

In some embodiments, the VOC is or comprises an alkane (e.g. C5-C20 alkane), an alcohol, a glycol, a terpene, a cyclic compound, an aromatic compound, a chlorinated aromatic compound, a nitro-aromatic compound, an aldehyde, a ketone, an amine, or an amide, including any combination thereof. In some embodiments, the VOC is a synthetic or a natural compound, characterized by the dipole moment as disclosed herein. In some embodiments, the VOC is characterized by a high boiling point of at least 70° C., at least 10° C., at least 150° C., at least 200° C., including any range between.

In some embodiments, the term “binding” including any grammatical form thereof refers to a non-covalent bond formation (such as hydrogen bonds, electrostatic interactions, Van-der-Waals bonds, dipol-dipol interactions, etc.). In some embodiments, the VOCs are adsorbed (e.g. physisorbed) to the polymeric fiber. In some embodiments, the VOCs are stably bound to the polymeric fiber. In some embodiments, the term “stable” refers to a substantial retention of the VOC content within the fibrous mat of the invention under ambient conditions (a temperature of less than 100° C., normal pressure or vacuum, and optionally ambient atmosphere), for a time period of between 1 day and 1 year including any range between.

In some embodiments, the fibrous mat (and/or the polymeric fiber) of the invention is characterized by enhanced binding affinity for the VOC, as compared to a control. In some embodiments, the binding affinity of the fibrous mat (and/or the polymeric fiber) of the invention for the VOC is enhanced by at least 2 times, at least 10 times, at least 100 times, at least 1000 times, at least 10.000 times, at least 1000.000 times, including any range between, as compared to a binding affinity for a control. In some embodiments, the nanoparticle of the invention is characterized by enhanced binding affinity for the VOC, as compared to a control, wherein a concentration of the VOC and of the control within the sample is substantially the same (e.g. less than 1000 ppm, less than 100 ppm, less than 10 ppm, less than 1 ppm, less than 1 ppb, less than 0.1 ppb, or between 0.01 ppb and 1 ppm, including any range between.

In some embodiments, the term “enhanced binding affinity” refers to an affinity ratio between (i) the binding affinity of the fibrous mat for the VOC and (ii) the binding affinity of the fibrous mat for the control. In some embodiments, the term “enhanced binding affinity” refers to adsorption efficiency for the VOC, as compared to the control. In some embodiments, the enhanced binding affinity refers to an enhanced adsorption (e.g. w/w ratio between the adsorbed VOC and the fibrous mat), as compared to the control. The w/w ratio between the adsorbed VOC and the fibrous may be deduced from the response intensity of a detector (e.g. a detector configured to detect the concentration of the VOC within the fibrous mat). In some embodiments, the affinity ratio of the fibrous mat, predetermines the selectivity of the fibrous mat to a specific VOC.

In some embodiments, the control refers to any gas (e.g. an atmospheric gas such as N2, CO2, water) and/or a VOC other than the specific VOC. In some embodiments, a concentration of the control within a given fluid sample (e.g. a gaseous or a liquid sample) is substantially the same or higher as the concentration of the analyte of interest such as less than 1000 ppm, less than 100 ppm, less than 10 ppm, less than 1 ppm, less than 1 ppb, less than 0.1 ppb, or between 0.01 ppb and 1 ppm, including any range between.

In some embodiments, the aromatic polymer is or comprises poly-diphenyl-phenylene oxide. In some embodiments, the aromatic polymer is or comprises poly-2,6-diphenyl-p-phenylene oxide.

In some embodiments, each polymeric fiber has cylindrical shape, or a ribbon-like shape. In some embodiments, the polymeric fibers within the porous membrane have the same or different shape. In some embodiments, the aromatic polymer is uniformly distributed along the entire cross-section of the fiber. In some embodiments, the polymeric fibers are characterized by a substantially uniform density along the entire cross-section of the fiber. In some embodiments, the polymeric fibers are substantially devoid of hollow-shape (or tube-like) fibers. In some embodiments, at least a portion of the polymeric fibers is in a form of a hollow fiber comprising a wall encapsuling a void core.

In some embodiments, the polymeric fibers are characterized by an average cross-section between 0.5 and 50 um, between 0.5 and 30 um, between 30 and 50 um, between 0.5 and 1 um, between 1 and 10 um, between 1 and 30 um, between 10 and 20 um, between 20 and 30 um, between 30 and 50 um, between 30 and 40 um, between 10 and 30 um, between 40 and 50 um, including any range between.

In some embodiments, the polymeric fiber is an electrospun fiber. In some embodiments, the polymeric fiber is an electrospun microfiber.

The term “electrospun” or “(electro)sprayed” when used in reference to polymers are recognized by persons of ordinary skill in the art and includes fibers produced by the respective processes. Such processes are described in more detail infra.

Methods for manufacturing electrospun elements as well as encapsulating or attaching molecules thereto are disclosed, inter alia, in WO 2014/006621, WO 2013/172788, WO 2012/014205, WO 2009/150644, WO 2009/104176, WO 2009/104175, WO 2008/093341 and WO 2008/041183.

Manufacturing of electrospun elements may be done by an electrospinning process which is well known in the art. Following is a non-limiting description of an electrospinning process. One or more liquefied polymers (i.e., a polymer in a liquid form such as a melted or dissolved polymer) are dispensed from a dispenser within an electrostatic field in a direction of a rotating collector. The dispenser can be, for example, a syringe with a metal needle or a bath provided with one or more capillary apertures from which the liquefied polymer(s) can be extruded, e.g., under the action of hydrostatic pressure, mechanical pressure, air pressure and high voltage.

The rotating collector (e.g., a drum) serves for collecting the electrospun element thereupon. Typically, but not obligatorily, the collector has a cylindrical shape. The dispenser (e.g., a syringe with metallic needle) is typically connected to a source of high voltage, preferably of positive polarity, while the collector is grounded, thus forming an electrostatic field between the dispenser and the collector. Alternatively, the dispenser can be grounded while the collector is connected to a source of high voltage, preferably with negative polarity. As will be appreciated by one ordinarily skilled in the art, any of the above configurations establishes motion of positively charged jet from the dispenser to the collector. Inverse electrostatic configurations for establishing motions of negatively charged jet from the dispenser to the collector are also contemplated.

At a critical voltage, the charge repulsion begins to overcome the surface tension of the liquid drop. The charged jets depart from the dispenser and travel within the electrostatic field towards the collector. Moving with high velocity in the inter-electrode space, the jet stretches and solvent therein evaporates, thus forming fibers which are collected on the collector, thus forming the electrospun element.

As used herein, the phrase “electrospun element” refers to an element of any shape including, without limitation, a planar shape and a tubular shape, made of one or more non-woven polymer fiber(s), produced by a process of electrospinning. When the electrospun element is made of a single fiber, the fiber is folded thereupon, hence can be viewed as a plurality of connected fibers. It is to be understood that a more detailed reference to a plurality of fibers is not intended to limit the scope of the present invention to such particular case. Thus, unless otherwise defined, any reference herein to a “plurality of fibers” applies also to a single fiber and vice versa. In some embodiments, the electrospun element is an electrospun fiber, such as electrospun fiber. As used herein the phrase “electrospun fiber” relates to a fibers formed by the process of electro spinning.

One of ordinary skill in the art will know how to distinguish an electrospun object from objects made by means which do not comprise electrospinning by the high orientation of the macromolecules, the fiber morphology, and the typical dimensions of the fibers which are unique to electrospinning.

The electrospun fiber may have a length which is from about 0.1 millimeter (mm) to about 20 centimeter (cm), e.g., from about 1-20 cm, e.g., from about 1-10 cm. According to some embodiments of the invention, the length (L) of the electrospun fibers of some embodiments of the invention can be several orders of magnitude higher (e.g., 10 times, 100 times, 1000 times, 10,000 times, e.g., 50,000 times) than the fiber's diameter (D).

Laboratory equipment for electrospinning can include, for example, a spinneret (e.g. a syringe needle) connected to a high-voltage (5 to 50 kV) direct current power supply, a syringe pump, and a grounded collector. A solution such as a polymer solution, sol-gel, particulate suspension or melt is loaded into the syringe and this liquid is extruded from the needle tip at a constant rate (e.g. by a syringe pump).

In some embodiments, parameters of the electrospinning process may affect the resultant substrate (e.g. the thickness, porosity, etc.). Such parameters may include, for example, molecular weight, molecular weight distribution and architecture (branched, linear etc.) of the polymer, solution properties (viscosity, conductivity & and surface tension), electric potential, flow rate, concentration, distance between the capillary and collection screen, ambient parameters (temperature, humidity and air velocity in the chamber) and the motion and speed of the grounded collector. Accordingly, in some embodiments, the method of producing a substrate as described herein includes adjusting one or more of these parameters.

In some embodiments, the polymeric fiber is a porous polymeric fiber comprising a plurality of pores. In some embodiments, the pores are in a form of perforations. In some embodiments, the pores are in a form of craters. In some embodiments, the porous polymeric fiber is characterized by average pore size of between 1 and 500 nm, between 1 and 10 nm, between 1 and 50 nm, between 10 and 50 nm, between 50 and 100 nm, between 1 and 100 nm, between 100 and 200 nm, between 200 and 300 nm, between 10 and 300 nm, between 300 and 500 nm, including any range between. A skilled artisan will appreciate that the average pore size may be calculated based on SEM micrographs of the matrix.

In some embodiments, a melting point (Tₘ) of the aromatic polymer within the polymeric fiber is lower than the Tₘ of the pristine polymer (e.g. in a form of particles) by at least 3° C., at least 5° C., at least 7° C., at least 10° C., including any range between. In some embodiments, the Tₘ of the aromatic polymer within the polymeric fiber is about 470° C.

In some embodiments, a glass transition point (Tg) of the aromatic polymer within the polymeric fiber is than the Tg of the pristine polymer by least 3° C., at least 5° C., at least 7° C., at least 10° C., at least 20° C., including any range between. In some embodiments, the Tg of the aromatic polymer within the polymeric fiber is about 223° C.

In some embodiments, a crystallinity degree the aromatic polymer within the polymeric fiber is reduced by at least 5%, at least 10%, at least 20%, at least 30%, at least 50%, at least 60%, as compared to the crystallinity degree of the pristine polymer, including any range between. In some embodiments, the aromatic polymer within the polymeric fiber is characterized by a crystallinity degree between 20 and 50%, between 20 and 30%, between 20 and 40%, between 30 and 50%, between 30 and 40%, between 40 and 45%, between 45 and 50%, including any range between. In some embodiments, the aromatic polymer within the polymeric fiber is characterized by a crystallinity degree of less than 50%, less than 48%, or less than 45%.

In some embodiments, the polymeric fiber is a solid at a temperature of up to 470° C., up to 400° C., up to 450° C., including any range between.

In some embodiments, an average molecular weight of the aromatic polymer is between 1.000 and 800.000 Da, between 1.000 and 200.000 Da, between 1.000 and 10.000 Da, between 10.000 and 100.000 Da, between 100.000 and 300.000 Da, between 100.000 and 200.000 Da, between 200.000 and 500.000 Da, between 100.000 and 500.000 Da, between 200.000 and 800.000 Da, between 300.000 and 800.000 Da, between 500.000 and 800.000 Da, including any range between.

In some embodiments, the term “average molecular weight” refers to a weight average molecular weight (Mw), which is well-known in the art. In some embodiments, the term “average molecular weight” refers to a number average molecular weight (Mn). In some embodiments, the term “Mn” generally refers to a molecular weight measurement that is calculated by dividing the total weight of all the polymer molecules in a sample with the total number of polymer molecules in the sample.

In some embodiments, the aromatic polymer constitutes up to 50%, up to 60%, up to 70%, up to 80%, up to 90%, up to 95%, up to 97%, up to 99%, up to 99.9%, up to 99.99%, or more by dry weight of the polymeric fibers, including any range between.

### Fibrous Mat

In one aspect of the invention, there is provided a fibrous mat comprising the polymeric fibers of the invention. In some embodiments, the fibrous mat comprises one or more (e.g. 2, 3, 4, 5, 6, or more) layers of the polymeric fibers. In some embodiments, the fibrous mat comprises a plurality of layers, wherein each layer is characterized by the same or different porosity, pore size, thickness, chemical composition and/or shape and/or surface morphology of the polymeric fibers, or a combination thereof.

In some embodiments, the fibrous mat further comprises a support layer bound to the one or more layers of the polymeric fibers. In some embodiments, the support layer is a porous layer, having substantial gas permeability. In some embodiments, the support layer is characterized by a gas permeability being the same greater than the gas permeability of the fibrous mat. In some embodiments, the support layer is or comprises a fibrous layer. In some embodiments, the support layer comprises a textile material (e.g. comprising woven, non-woven, synthetic or natural fibers).

In some embodiments, the fibrous mat is characterized by air permeability coefficient of at least 10⁻¹⁴ m², at least 2*10⁻¹⁴ m², at least 5*10⁻¹⁴ m², at least 10⁻¹³ m², at least 10⁻¹² m², at least 2*10⁻¹² m², at least 5*10⁻¹² m², at least 7*10⁻¹² m², at least 10⁻¹¹ m², including any range between. In some embodiments, the fibrous mat is characterized by air permeability coefficient of between 10⁻¹⁴ and 10⁻¹¹ m², between 10⁻¹⁴ and 5*10⁻¹⁴ m², between 5*10⁻¹⁴ and 10⁻¹³ m², between 10⁻¹² and 5*10⁻¹³ m², between 5*10⁻¹³ and 10⁻¹² m², between 10⁻¹² and 5*10⁻¹² m², between 5*10⁻¹² and 10⁻¹¹ m², including any range between. In some embodiments, the fibrous mat is in a form of a gas sampling membrane, and wherein a pressure drop of the mat of the invention is the same or lower than the pressure drop of an analogous stationary phase column composed of the same amount of Tenax beads.

In some embodiments, the fibrous mat is or comprises a non-woven uniform layer or matrix, wherein matrix is as described hereinabove.

In some embodiments, the fibrous mat is a porous matrix, characterized by an average porosity of about 50%, about 70%, about 90%, or more, including any range between. In some embodiments, the fibrous mat is characterized by a porosity of between 70 and 90%, between 70 and 75%, between 75 and 80%, between 80 and 85%, between 70 and 85%, between 85 and 90%, between 70 and 95%, between 90 and 95% including any range between.

In some embodiments, the fibrous mat comprises a single layer or a plurality of layers. In some embodiments, the fibrous mat comprises a plurality of layers wherein each layer has a distinct thickness, a distinct chemical composition, or any other physico chemical property such as porosity, pore size, density, etc. In some embodiments, the plurality of layers are the same layers.

In some embodiments, the fibrous mat is characterized by an average pore size ranging between 5 and 100 um, between 5 and 50 um, between 5 and 10 um, between 10 and 30 um, between 30 and 50 um, between 50 and 80 um, between 80 and 100 um, or greater than 100 um, including any range between. In some embodiments, the fibrous mat is characterized by an average pore size of 5 micron or more. In some embodiments, the fibrous mat is characterized by an average pore size of less than 50 um. In some embodiments, the pore size of the fibrous mat refers to a void space between the polymeric fibers within the matrix. A skilled artisan will appreciate that the average pore size may be calculated based on SEM micrographs of the matrix.

In some embodiments, the fibrous mat is in a form of a self-supporting membrane. As used herein, the term “self-supporting” means that the membrane can hold a definable shape in the x-, y-, and z-plane in the absence of any applied force or in the absence of any supporting substrate or polymer. That is, in some embodiments, the disclosed membrane is devoid of supporting substrate or polymer. In some embodiments, the membrane is characterized by a sufficient tensile strength as described below. In some embodiments, the mat of the invention has a sufficient mechanical strength and is characterized by sufficient elasticity suitable for utilizing thereof as a membrane (e.g. a membrane configured for supporting gas flow at a flow rate/velocity a described herein).

In some embodiments, the fibrous mat is characterized by an average a thickness of between 20 and 2000 um, between 50 and 2000 um, between 20 and 50 um, between 50 and 100 um, between 100 and 200 um, between 100 and 500 um, between 500 and 1000 um, between 100 and 2000 um, between 200 and 2000 um, between 500 and 2000 um, between 1000 and 2000 um, including any range between.

In some embodiments, the fibrous mat is characterized by a cross-section between 0.1 and 50 cm, between 0.1 and 1 cm, between 1 and 5 cm, between 5 and 10 cm, between 10 and 50 cm, including any range between. In some embodiments, the fibrous mat is characterized by a cross-section about 1 m, or more.

In some embodiments, fibrous mat is characterized by a density of between 0.01 and 0.2 gr/cm³, between 0.01 and 0.1 gr/cm³, between 0.01 and 0.07 gr/cm³, between 0.02 and 0.07 gr/cm³, between 0.02 and 0.2 gr/cm³, between 0.02 and 0.05 gr/cm³, between 0.05 and 0.2 gr/cm³, between 0.05 and 0.1 gr/cm³, between 0.1 and 0.2 gr/cm³, including any range between.

In some embodiments, the fibrous mat is characterized by a BET surface area greater than the BET of the pristine polymer by at least 10%, at least 10%, at least 20%, at least 30%, at least 50%, at least 60%, including any range between.

In some embodiments, the fibrous mat is characterized by a BET surface area of at least 25 m²/g, at least 30 m²/g, at least 50 m²/g, at least 60 m²/g, at least 70 m²/g, at least 100 m²/g, including any range between.

In some embodiments, the fibrous mat is characterized by a tensile strength of at least 0.4 MPa, at least 1 MPa, at least 5 MPa, at least 10 MPa, at least 50 MPa including any range between.

In some embodiments, the fibrous mat is characterized by elongation to break of between 5 and 10%, between 5 and 7%, between 7 and 10%, including any range between.

In some embodiments, the fibrous mat is characterized by Young's modulus between 5 and 10 MPa, between 6 and 9 MPa, between 5 and 7 MPa, between land 10 MPa, including any range between.

In some embodiments, the fibrous mat is configured to support a flow of a fluid. In some embodiments, the fibrous mat is configured to support gas flow at a flow velocity of at most 5 m/sec. In some embodiments, the fibrous mat is configured to support gas flow at a flow velocity between 0.05 and 5 m/sec, between 0.05 and 0.1 m/sec, between 0.1 and 0.5 m/sec, between 0.5 and 1 m/sec, between 1 and 3 m/sec, between 3 and 5 m/sec, including any range between.

In some embodiments, the fibrous mat is configured to support gas flow at a flow rate of at least 1 L/min, at least 10 L/min, at least 50 L/min, at least 100 L/min, at least 200 L/min, at least 300 L/min, including any range between, wherein the dimensions (thickness and cross-section) of the mat are as described herein.

In some embodiments, the fibrous mat is characterized thermal stability at a temperature of up to 500° C., up to 490° C., up to 470° C., or up to 450° C., including any range between, wherein the thermal stability is as described herein.

In some embodiments, the mat is configured for sampling VOC from a fluid sample. In some embodiments, the fluid sample is a gas or a liquid. In some embodiments, the mat has a binding affinity to the VOC, as described hereinabove. In some embodiments, sampling is by a dynamic sampling (e.g. via passing a flowing sample through the membrane), or by a static sampling (e.g. by exposing the membrane to the sample), as disclosed herein. In some embodiments, the terms “fibrous mat” and “mat” are used herein interchangeably.

In some embodiments, the mat is substantially dry. In some embodiments, the mat is substantially devoid of moisture. In some embodiments, the mat of the invention comprises trace amounts of any one of DMSO, DMF, or any other high boiling polar solvent, or any combination thereof. In some embodiments, the mat of the invention is substantially devoid of trace amounts of an organic solvent.

In some embodiments, the mat is capable of adsorbing a sufficient amount of the VOC. In some embodiments, the sufficient amount is so as to allow detection of the VOC by a detector. In some embodiments, the sufficient amount (or sorption capacity) comprises at a w/w ratio between the VOC and the mat up to 1:10, or between 1:1.000.000 and 1:10, between 1:1.000.000 and 1:100.000, between 1:100.000 and 1:10.000, between 1:20.000 and 1:1000, between 1:1000 and 1:500, between 1:500 and 1:100, between 1:100 and 1:50, between 1:50 and 1:10, including any range between.

In some embodiments, the mat is characterized by a DSC pattern exhibiting at least one endothermic peak in the range from 470° C. to 500° C., or from 490 to 500° C. An exemplary DSC pattern of the mat is represented herein.

In another aspect, there is provided an article comprising the plurality of polymeric fibers of the invention and/or the mat disclosed herein. In some embodiments, the article is a VOC sampler. In some embodiments, the article comprises the mat and/or the polymeric fibers embedded within a container (e.g. housing). In some embodiments, the housing defines a lumen and further comprises at least 2 openings (e.g. an inlet and outlet). In some embodiments, the housing is configured to receive a fluid volume or a gas volume of the sample. In some embodiments, the article is in operable communication with a fluid sample. In some embodiments, the article is configured to support a flow of the fluid sample through the lumen (e.g. along a longitudinal axis of the lumen). In some embodiments, the article is configured to support a flow of the fluid sample through the membrane. In some embodiments, the article is configured to be in operable communication with a pump.

In some embodiments, the article is a VOC sorbent. In some embodiments, the article is a VOC sampler. In some embodiments, the article is configured for sampling one or more VOCs from a fluid sample (e.g. a gas and/or a liquid). In some embodiments, the article is in a form of a gas sampler. In some embodiments, the article is in a form of a liquid sampler. In some embodiments, the article is a filter. In some embodiments, the article is a gas filter. In some embodiments, the article is a liquid filter.

### Method of Manufacturing

In another aspect, there is provided a method for manufacturing the fibrous mat of the invention, the method comprises forming a polymeric solution comprising the aromatic polymer disclosed herein and a solvent; and introducing (e.g. by injection) the polymeric solution into an electrospinning apparatus under appropriate conditions, thereby obtaining the fibrous mat of the invention. In some embodiments, the appropriate conditions comprise conditions appropriate for inducing electrospinning of the polymeric solution, thereby fabricating the electrospun polymeric fibers of the invention.

For electrospinning, the polymeric solution is injected into the electrospinning apparatus to obtain the electrospun polymeric fibers as disclosed herein. In some embodiments, the method of fabricating the polymeric fibers and/or the mat of the invention comprises forming a polymeric solution by dissolving a sufficient amount of the aromatic polymer disclosed herein within an appropriate solvent. In some embodiments, the solvent of the polymeric solution is characterized by a sufficient electrical conductivity, required for electrospinning process. In some embodiments, the solvent comprises one or more solvents. In some embodiments, the solvent comprises at least one polar solvent such as DMF, DMSO, an alcohol (e.g. ethanol, methanol, etc.). In some embodiments, the polar solvent is characterized by a dielectric constant of at least 15, at least 20, at least 25, at least 30, or more including any range between.

In some embodiments, the solvent of the first polymeric solution comprises a chlorinated solvent (such as chloroform, DCM, dichloroethane, etc.) and a polar solvent (e.g. DMSO, ethanol or both). In some embodiments, a v/v ratio between the chlorinated solvent and the polar solvent within the polymeric solution is between 7:3 and 9.5:0.5, between 7:3 and 8:2, between 8:2 and 9:1, between 8:2 and 8.5:1.5, between 8.5:1.5 and 9:1, between 8.5:1.5 and 9.5:0.5 including any range between.

In some embodiments, a w/w concentration of the aromatic polymer within the polymeric solution is between 0.1 and 20%, between 0.1 and 1%, between 1 and 10%, between 1 and 5%, between 5 and 20%, between 5 and 10%, between 10 and 20%, including any range between.

In some embodiments, forming a polymeric solution comprises dissolving a sufficient amount of the aromatic polymer within the chlorinated solvent to obtain a solution, and subsequently adding the polar solvent to the solution at a sufficient amount, thereby obtaining the polymeric solution; wherein the sufficient amount is so as to obtain a predetermined v/v ratio between the chlorinated solvent and the polar solvent within the polymeric solution.

In some embodiments, the polymeric solution is injected into the electrospinning apparatus at a predetermined flow rate. In some embodiments, the flow rate and/or the concentration of the polymeric solution predetermine the fiber cross-section and surface porosity. Furthermore, inter-fiber pores within the mat can be manipulated by varying the composition of the polymeric solution and the electrospinning parameters Non-limiting exemplary electrospinning conditions are as disclosed in any one of WO2008/041183, WO 2009/104174, WO 2009/104176 the contents of which are incorporated herein in their entirety. Exemplary electrospinning conditions are disclosed in the Examples section.

In some embodiments, the method further comprises exposing the fibrous mat of the invention to a temperature of above 100° C., above 200° C., above 250° C., above 270° C., above 300° C., including any range between. In some embodiments, the method further comprises exposing the fibrous mat of the invention to a temperature of at most 350° C., at most 300° C., at most 290° C., at most 270° C., at most 250° C., at most 200° C., including any range between. In some embodiments, the method further comprises exposing the fibrous mat of the invention to a temperature disclosed herein for a time period sufficient for annealing of the fibrous mat.

### Method for Sampling/Analyte Detection

In another aspect, there is provided a method for sampling a VOC from a fluid sample, the method comprises contacting the fibrous mat of the invention with the fluid sample under appropriate conditions, thereby obtaining the VOC adsorbed to the fibrous mat. In some embodiments, the fluid sample is a gaseous sample or a liquid sample. In some embodiments, the fluid sample comprises one or more VOCs.

In some embodiments, contacting comprises exposing the fibrous mat to the fluid sample (e.g. by providing the fibrous mat in close proximity to the gaseous sample). In some embodiments, contacting comprises exposing the fibrous mat to a flowing fluid sample. In some embodiments, the flowing fluid sample is characterized by a flow rate between 50 ml/min and 20.000 L/min, between 50 and 100 ml/min, between 100 and 200 ml/min, between 100 and 2000 ml/min, between 200 and 1000 ml/min, between 200 and 500 ml/min, between 500 and 2000 ml/min, between 500 and 1000 ml/min, between 1000 and 2000 ml/min, between 1 and 10 L/min, between 1 and 20.000 L/min, between 1 and 100 L/min, between 100 and 1.000 L/min, between 1000 and 10.000 L/min, between 10.000 and 20.000 L/min, including any range between.

In some embodiments, the flowing fluid sample is characterized by a flow velocity of between 0.05 and 5 m/sec, between 0.05 and 0.1 m/sec, between 0.1 and 0.5 m/sec, between 0.5 and 1 m/sec, between 1 and 3 m/sec, between 3 and 5 m/sec, including any range between.

In some embodiments, contacting is performed under appropriate conditions comprising a sampling time sufficient for adsorbing an amount of the VOC sufficient for detection by a detector. In some embodiments, sufficient amount of the VOC is as described herein. In some embodiments, sufficient amount refers to the sorption capacity of the mat.

In some embodiments, the sampling time is between 1 second (s) and 10 hours (h), at least 1 second (s), at least 2 s, at least 5 s, at least 8 s, at least 10 s, at least 30 s, at least 60 s, at least 1.5 minute (min), at least 2 min, at least 3 min, at least 5 min, or between 10 s and 10 min, between 10 s and 60 s, between 1 and 2 min, between 1 and 10 min, between 2 and 5 min, between 5 and 10 min, between 10 and 60 min, between 1 and 3 h, between 3 and 10 h, including any range between.

In some embodiments, appropriate conditions further comprise an operable temperature between −10 and 100° C., between 0 and 10° C., between 0 and 25° C., between 10 and 30° C., between 10 and 20° C., between 10 and 40° C., between 20 and 30° C., between 30 and 40° C., between 40 and 50° C., between 50 and 100° C., including any range between.

In some embodiments, appropriate conditions further comprise a pressure between 0.0001 and 100 bar, a relative humidity of between 0 and 100%, between 10 and 100%, between 0 and 10%, between 10 and 30%, between 30 and 100%, between 40 and 100%, between 40 and 60%, between 40 and 70%, between 40 and 80%, between 60 and 80%, between 80 and 100%, including any range between.

In another aspect, there is provided a method for detecting an analyte of interest within a sample, the method comprising sampling a VOC from the sample as described hereinabove, thereby obtaining the VOC adsorbed to the fibrous mat; and subsequently exposing the VOC adsorbed to the fibrous mat to a detector, thereby determining the presence of the analyte of interest within the sample. In some embodiments, the analyte of interest is a VOC, wherein the VOC is as described hereinabove. In some embodiments, the detector is configured for detecting the presence and/or the concertation of the analyte of interest adsorbed to the fibrous mat.

In some embodiments, the sample is a fluid sample (e.g. a gaseous sample or a liquid sample).

In some embodiments, exposing step comprises desorbing the VOC from the fibrous mat. In some embodiments, desorbing comprises contacting to the fibrous mat with a sufficient amount of an appropriate solvent, thereby obtaining a solution comprising the VOC. In some embodiments, the appropriate solvent is capable of dissolving the VOC.

In some embodiments, desorbing comprises a thermal desorption by exposing the fibrous mat to a temperature in a range between 50 and 300° C.

### General

As used herein the terms “about” and “approximately” refers to ±10%.

The terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, electrochemical, and electronical arts.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### EXAMPLES

The inventors successfully manufactured fibrous membranes comprising Tenax-based electrospun polymeric fibers with tunable morphology and geometry via electrospinning process. The morphology of the membrane, specifically, fiber's size, surface porosity and the inter-fiber pores has been modified by varying the solution composition and the electrospinning parameters. Various electrospun membranes can be designed and produced in a diversity of geometries depending on the designated application (dynamic or static sampling, high/low flow rates, environments sampling or biomarkers sampling (skin patches)). Exemplary electrospun membranes of the invention exhibited greater specific surface area and were characterized by enhanced adsorption efficiency in comparison to commercial Tenax granules. Exemplary electrospun membranes of the invention have been implemented for both, static and dynamic sampling. Additionally, the inventors successfully detected up to 0.1 ppb numerous VOCs (e.g. natural terpenes, aromatic compounds, and/or explosives) within a gaseous sample, by implementing exemplary electrospun membranes of the invention together with analytical detectors such as ion mobility spectrometer, and thermal desorption mass spectrometer.

### Materials and Methods

Tenax-TA beads (Buchem BV) were purchased from Sigma, Ltd. Chloroform, Dimethyl sulfoxide (DMSO) and Ethanol were supplied by Merck.

Preparation of Fibrous Membranes by Electrospinning Process:

Tenax solution (6.3% wt) was prepared by dissolving Tenax beads in a mixed solvent systems: chloroform:DMSO and chloroform:DMSO:EtOH at various solvent's ratios, in a two-step protocol. First, the TENAX beads were dissolved in chloroform, then the non-solvent DMSO/EtOH were added to obtain stable electrospinning process. The solution was electrospun at a flow rate of 1-2 ml/hr, a voltage of 8-10 kV, and at a needle to collection drum distance of 9 cm. Fibers were collected on the rotating drum into a non-woven fabric.

### Example 1

### Preparation of Tenax-Based Electrospun Fibrous Membranes

The inventors found that TENAX polymer has high solubility in chlorinated solvents (e.g., chloroform), however, due to relatively low dielectric constant of chlorinated solvents (such as chloroform) they are less suitable for electrospinning. Accordingly, in order to enhance conductivity of the polymeric electrospinning solution, the inventors utilized one or more co-solvents (also used herein as the “polar solvent”) with high dielectric constant, such as DMSO and ethanol. Surprisingly, it was observed that the addition of the co-solvent(s) significantly improved the spinnability of TENAX solution resulting in stable spinning process. Nevertheless, both co-solvents, DMSO and ethanol act as non-solvents for TENAX polymer, i.e., their addition may alter the solvent quality to TENAX polymer, leading to phase separation. The inventors tested various solvent compositions and/or solvent ratios so as to arrive at preferable solvent composition appropriate for a stable electrospinning process and for the formation of polymeric fibers of the invention with predefined shape and/or surface morphology.

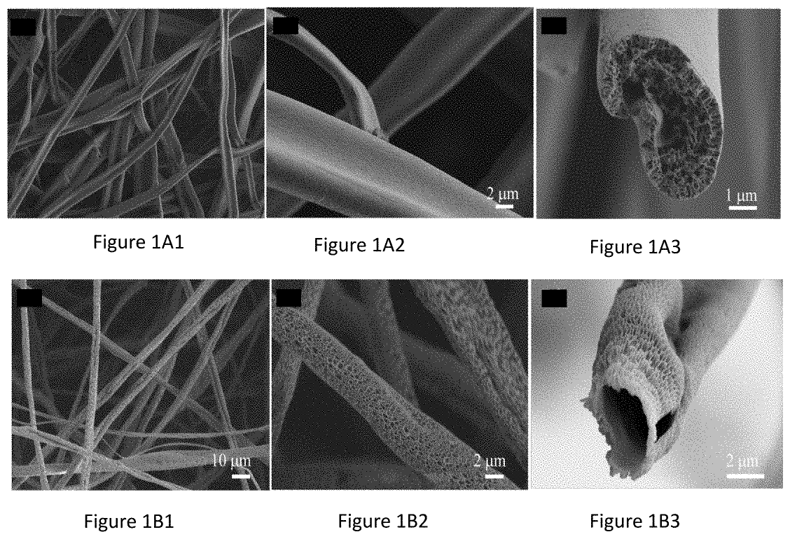

The shape and morphology of non-limiting exemplary fibrous mats of the invention such as in a form of electrospun membranes (ESM) are presented in FIG. 1. The images present fibers electrospun from Tenax solutions with various solvent composition: chloroform:DMSO and chloroform:DMSO:EtOH. The fibers electrospun from chloroform:DMSO (86%:14% v/v) displayed flat, ribbon structure with smooth surface. The cross-section of these fibers revealed interior porosity (Fig. A1-3). Exemplary solvent compositions and physical properties of the exemplary electrospun membranes are summarized in Table 1 below.

TABLE 1
Electrospun membranes (ESM) obtained from different
electrospining solutions, fiber characterization
and spesific surface area obtaibed by
BET analysis BET, compared to commercial Tenax beads.

| | | | | BET |
|---|---|---|---|---|
| | | | Fiber | surface |
| | Electrsopining | | diameter, | area, |
| Sample | soluion | Composition | mm | m²/gr |
| ESM-A | Chloroform/DMSO | 86%:14% | 5.8 ± 0.94 | 70 |
| ESM-B | Chloroform/ | 83%:7%:10% | 3.2 ± 0.52 | 52 |
| | DMSO/EtOH | | | |
| ESM-C | Chloroform/ | 86%:7%:7% | 5.4 ± 1.02 | |
| | DMSO/EtOH | | | |
| ESM-D | Chloroform/ | 80%:7%:13% | 3.7 ± 0.93 | 59 |
| | DMSO/EtOH | | | |
| Beads | | | | 24.6 |

Using ethanol as co-solvent resulted in a substantially different fiber's morphology. At a concentration of chloroform/ethanol 90/10 (v/v) fibers with extremely porous surface, in the range of 300 nm in diameter were formed. Nevertheless, the spinning process was unstable thus DMSO was included in the solvent system. The electrospinning of ternary solvent system chloroform/DMSO/ethanol yielded porous surface and hollow interior (FIG. 1B 1-3).

The fibers electrospun from triple solvent system chloroform/DMSO/EtOH exhibit porous surface, while the surface porosity and the interior morphology was dependent on the solvents ratio within the mixed solution. Extremely porous surface (50-300 nm pore size) and hollow interior was obtained in case of chloroform/DMSO/EtOH (83%:7%:10% v/v), while in case of chloroform/DMSO/EtOH (86%:7%:7% v/v) smaller pores on the surface were obtained (FIG. 1C). The size of the fibers and the inter-fiber distance was also composition dependent.

Without being limited to any particular theory, it is postulated, that a mechanism responsible for the internal/surface porosity formation is non-solvent induced phase separation (NIPS). It is postulated, that in case of chloroform/DMSO solvent system, while the chloroform rapidly evaporates from the jet surface, the DMSO portion increase within the interior of the fiber due to its significantly lower volatility. It is further postulated, that different evaporation rates of the chlorinated solvent and of the polar solvent result in the formation of non-solvent regions, which in case of DMSO accumulates within the fiber and form internal porosity, while in case of ethanol diffuses form on the surface forming surface porosity.

The porosity was obtained via non-solvent induced phase separation (NIPS) which occurs when non-solvent as DMSO or ethanol are added to solvent-polymers system. Additional morphologies were obtained by further changing the solvent composition, see FIG. 1D.

### Specific Surface Area (BET)

The specific surface area of the samples was estimated using Brunauer-Emmett-Teller (BET) analysis. The measured surface area of ESM showed about 3 fold increase in specific surface area in comparison to the granular form (see Table 1). The largest surface area was obtained for the fibers having internal porosity, suggesting the interior pores are accessible to the adsorbate (e.g. a VOC disclosed herein).

### TGA and DSC

Thermal stability is crucial for adsorbent material particularly when the adsorbent is designated for thermal desorption uses (TD-GC-MS). The thermal properties of the electrospun fibers were characterized and compared to those of commercial granular Tenax.

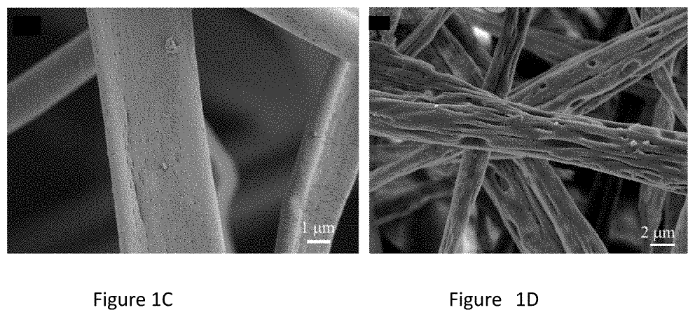

The thermogravimetric (TG) curves, which describe the thermal behavior of commercial Tenax beads and EMS-A, are given in FIG. 2. The main degradation peak was observed for both samples, beads and membrane, at 505° C., which is assigned to thermal decomposition of the polymer. These results indicated that the thermal stability of Tenax polymer was not degraded by the electrospinning process.

It has been shown in the literature that Tenax films with higher porosity and lower crystallinity displayed higher adsorption capacity. Thus, the inventors performed Differential scanning calorimetry (DSC) analysis to determine thermal transitions and crystallinity degree of the as spun membranes in comparison to commercial beads.

The electrospun membranes exhibit reduced glass transition temperature, Tg, and lower degree of crystallinity in comparison with Tenax beads. The crystallinity of the as spun fibers was considerably lower than the crystallinity of the beads (30% vs. 58% respectively). It should be noted that the as spun electrospun fibers exhibit cold crystallization peak in the temperature range of 261-264° C., accordingly the “as spun” membranes underwent annealing at 270° C., for subsequent use thereof in a thermal desorption analysis.

### Permeability of the Electrospun Fibers

Permeability is an important property of membranes, specifically when dynamic sampling is performed. Permeability is a measure of how easily a fluid passes through the filter medium and is defined by Darcy's law:

$$Q = \frac{kA}{μ} \frac{Δ P}{L} ,$$

where Q is the air flow rate, A is the filter area, k is the permeability, μ is the air dynamic viscosity, ΔP is the pressure drop, and L is the thickness of the filter medium. Darcy's equation demonstrates the normal linear dependence of flow rate with pressure drop for low Reynolds number laminar flows.

The permeability was measured using custom-made instrument. An air flow was applied through the membrane at a flow rate of Q=0.5-5 L/hr and a subsequent pressure drop (dP) was measured. The Q vs. dP/dL data was plotted and the permeability parameter (k) was extracted according to Darcy law.

The permeability values (k) of ESM samples are listed in Table 2. The results indicate that air permeability is affected by structural characteristics of the membranes, namely fibers size and inter-fiber distance. The k values of ESM A-C were comparable, while the k of ESM-D, (grooved fibers, fluffy membrane) was one order of magnitude higher.

It should be noted that the permeability of electrospun membranes is about 1-2 orders of magnitude lower in comparison to commercial column, mainly because of the denser structure. i.e., smaller inter particle distance. the performance of a filter is usually estimated by its pressure drop across the filter.

The performance of a filter is usually estimated by its pressure drop across the filter. According to Darcy's low the pressure drop is

$$Δ P = ( \frac{Q}{A} ) ( \frac{μ}{k} ) L ,$$

proportional to the media thickness (L) and inversely proportional to cross-section of the media (A). The characteristic thickness of ESM is about 0.3 mm, which is two orders of magnitude smaller in comparison to the column bead length (L˜30 mm). Thus, the pressure drop for Tenax ESM is comparable or lower in comparison to conventional Tenax bead column. Moreover, A is a controllable feature of the membrane, and can be adjusted during the spinning process. The cross-section area can be enlarger at by 300 in comparison to cross-section of the commercial TENAX column, which in turn lowers the pressure drop.

TABLE 2
Summary of air permeability values of Tenax ESM and
commercial column bead.

| | |
|---|---|
| ESM_A | 2.63E−13 ± 6.12E−14 |
| ESM_B | 5.85E−13 ± 6.03E−14 |
| ESM_C | 2.68E−13 ± 7.78E−13 |
| ESM_D | 1.25E−12 ± 7.40E−13 |
| Column (Tenax Beads) * | 1-50E−11 |

### Example 2

### Adsorption Efficiency of Tenax-Based Electrospun Fibrous Membranes

Evaluation of adsorption efficiency of Tenax ESM using static and dynamic sampling has been performed.

### Static Sampling

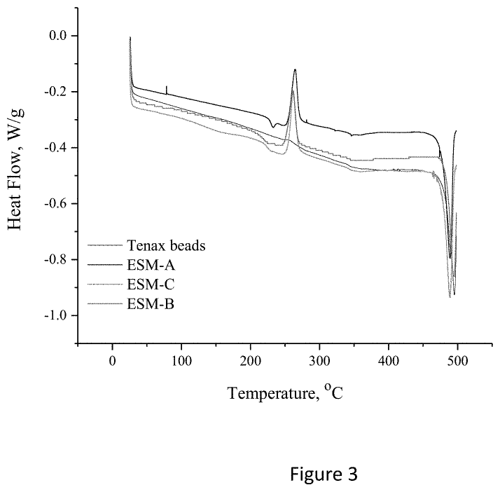

Measurements of static adsorption by Tenax ESM and beads were conducted in a 5 L glass desiccator. The analytes of interest (Musk Xylene, Naphthalene, Thymol) were deposited on the bottom of the vessel (in excess ˜5 gr), to obtain a vapor pressure of the analyte in the vessel. The samples were mounted on a support grid and the desiccator was sealed. The amount of analyte adsorbed on Tenax ESM and Tenax beads, after 3 hr, was measured using solvent extraction method and subsequent GC-MS analysis. The results show enhanced adsorption capacity of TENAX ESM in comparison to Tenax granules, for all analytes tested (FIG. 4A). This superior performance of electrospun fibers in comparison to TENAX-beads is ascribed to larger surface area of the former and more accessible fiber's surface towards the adsorption of VOC. The vapor pressures (in atm) of the tested VOCs Musk Xylene, naphthalene, and thymol are 10⁻⁹, 10⁻⁴, and 2*10⁻⁵, respectively.

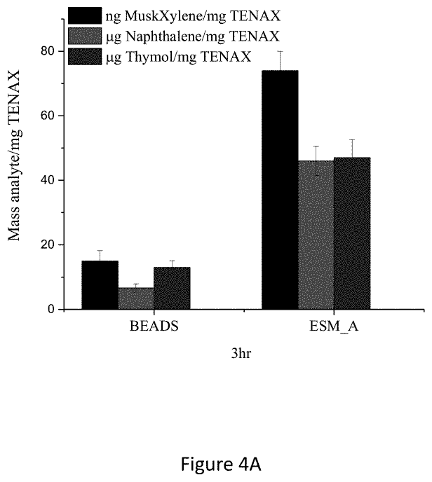

Additionally, the adsorption efficiency of different ESM was studied using naphthalene. The highest adsorption amount was obtained for electrospun fibers type ESM-A, which possess smooth surface and porous interior. Surprisingly, the fibers with high surface porosity demonstrated reduced adsorption capacity suggesting the adsorbate utilize not only the outer surface area (on the surface of the fibers) but also the interior surface. FIG. 5 demonstrates the enhanced adsorption kinetics, along with higher equilibrium values of Musk Xylene analyte.

### Dynamic Sampling

The dynamic sampling efficiency of ESM in comparison to traditional packed column with Tenax beads was studied. It should be noted that according to manufacturer, sampling using Tenax column is restricted to a flow rate of 0.5-1 L/min, while in case of electrospun membrane flow rates as high as 200 L/min were applied. Herein the inventors successfully performed fast pre-concentration of a VOC of interest (Musk xylene) at a concentration as low as 0.1 ppb within a gaseous sample.

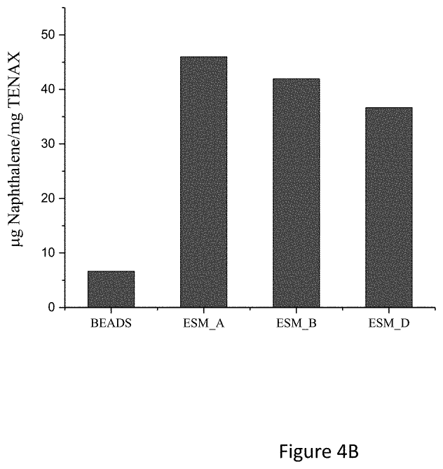

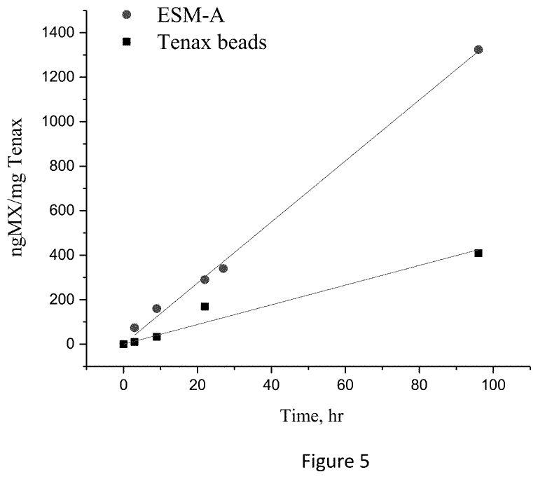

In one experiment, the air sample was created by coating the walls of a glass bubbler with musk xylene crystals. The bubbler was held inside a hot bath with constant temperature of 26 deg C. The analyte concentration within the bubbler, as measured, was ˜10 ppb. The commercial Tenax tube (FIG. 6B) and the Tenax ESM mounted inside costume housing (FIG. 6C) were connected, in turns, to the bubbler outlet. The headspace of the bubbler was sampled with both filters at different flow rates. It was found that the Tenax ESM exhibit about 5 to 10 fold higher adsorption (ng Musk/mg Tenax*min) in comparison to commercial column packed with Tenax beads. This effect is attributed to both the enhanced specific surface area of the fibers and the larger cross-section of the membrane itself. In addition, it was found that the resistance to flow presented by the column was significantly higher than the membranes.

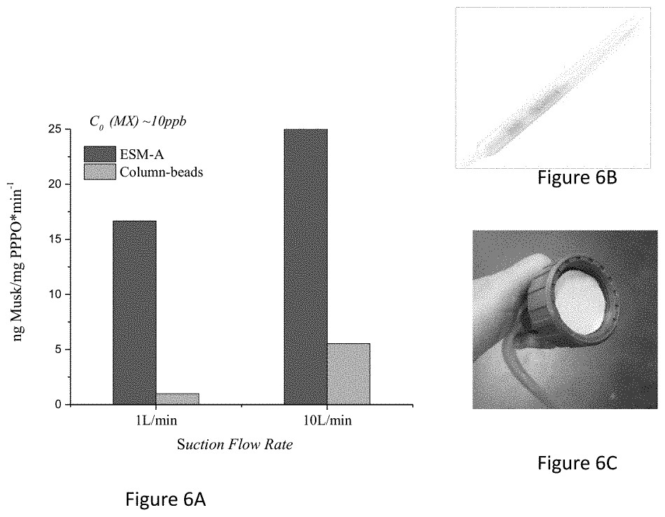

In another experiment, the air sample was created by inserting about 300 gr of musk xylene into a large metal container (˜34 m³ free volume) and keeping it closed for more than 7 days. The concentration of musk xylene in the indoor air reached equilibrium concentration of about 1 ng/L (0.1 ppb). After reaching equilibrium, the indoor air was sampled with the commercial Tenax column and with the Tenax ESM (A=5 cm²) at flow rates of 1, 10 and 100 L/min. The total musk xylene adsorbed on both filters was measured by solvent desorption using acetone and then GC-MS analysis. It was found that for every sampling flow rate, the Tenax ESM exhibit about 10 fold higher adsorption rate (ng Musk/mg Tenax*min) in comparison to commercial column packed with Tenax beads. Furthermore, it was found not feasible to sample the air with the commercial column at flow rate of 100 L/min, whereas the ESM of the invention has been successfully operated at even greater flow rates (FIG. 7).

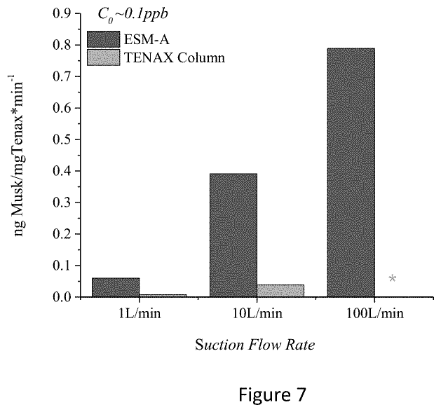

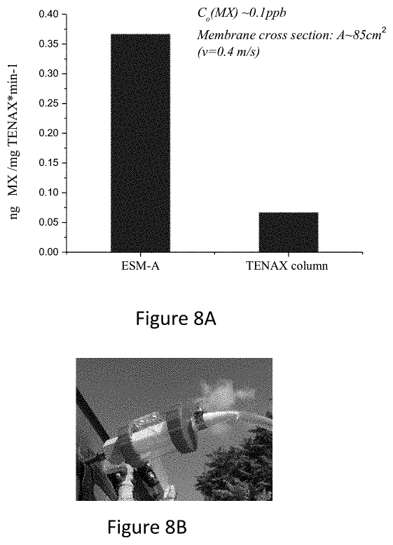

In a field test experiment, a 6-feet marine container was loaded with a 12 kg of musk xylene and packed with cardboard boxes (˜10 m³ free volume). The container was kept closed for 7 days, so as to obtain the interior air composition in an equilibrium. Subsequently, the interior air of the closed container was sampled via a 8 mm SS tubing (i) with the commercial Tenax column, and (ii) with the custom-made filter containing an exemplary porous membrane of the invention having a similar amount of Tenax as in the column (˜150 mg) and a cross-section (A) of 85 cm² (FIG. 8B). For each sampling apparatus, the sampling flow rate was set to the maximum possible ˜10 L/min for the column and 200 L/min for the membrane. Each apparatus sampled the container for 30 min. After analysis by GC-MS it was found that the total amount of the VOC (musk xylene) adsorbed onto the ESM was about five fold larger than the commercial Tenax column.

Accordingly, it should be apparent that exemplary fibrous mats of the invention are characterized by superior sampling capability (or adsorption efficiency) compared to a similar column comprising Tenax beads and that the fibrous mats of the invention can be implemented for sampling of fluid samples with trace amounts of VOCs of interest.

### Example 3

### Use of Tenax-Based Electrospun Fibrous Membranes for Sampling and Subsequent Detection of Trance Nitro-Aromatic Compound (TNT)

Tenax ESM has been used for sampling and detection of 2,4,6 trinitrotoluene (TNT) in an air sample. A portable Ion Mobility Spectrometer (IMS)-MMTD was used as a detector.

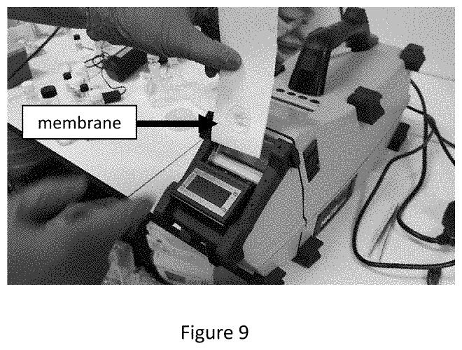

In this experiment, few milligrams of TNT adsorbed on cotton cloth, were placed in a 250 ml glass jar open to room air. Then, the headspace of the jar has been sampled by positioning Tenax ESM on the suction head and by applying vacuum for 1 minute at a suction speed of 3 L/min. Subsequently, the Tenax ESM was disassembled and placed inside an adaptor that enabled inserting it into the inlet of the IMS (FIG. 9). A readout of the instrument indicated the presence of TNT within the sample.

### Example 4

### Use of Tenax-Based Electrospun Fibrous Membranes for Sampling and Subsequent Detection of Human Breath Volatiles

The inventors further demonstrate the feasibility of direct coupling of the fibrous mats of the invention (e.g. in a form of a membrane) with standard analytical detectors. For the purpose of the demonstration, the inventors chose to use a portable Ion Mobility Spectrometer (IMS)-MMTD as an exemplary detector. This device has a built-in thermal disrober which is specifically designed to vaporize chemicals trapped as particles or adsorbed on a designated swab. The chemical vapors are then separated and measured. The IMS is a rapid analytical technique and allows detection of up to nanograms of specific chemical components present in a matrix within a few seconds. This quality makes the technology suitable for various applications such as portable explosives detectors or point-of-care instruments for medical applications.

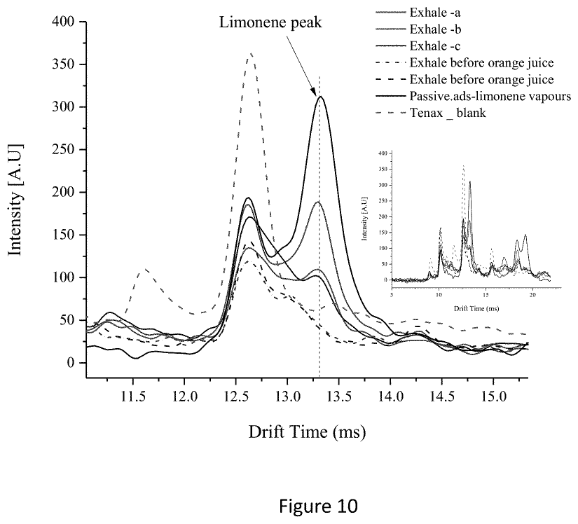

Limonene is a VOC present in many natural and processed foods, cosmetics, and cleaning products. Although Limonene is not a product of the human metabolic process it was found to be present in the breath composition of humans in a broad range of concentrations. Specifically, the high presence of limonene in the breath can indicate a disorder in liver functions. In this experiment, the inventors demonstrate a proof of concept for rapid breath collection analysis for the detection of limonene in human breath. Breath was collected by using a Tenax ESM (1-inch in diameter). The membrane was mounted inside a record connector (¾″×25 mm, Plasson) and breath was exhaled through it. After the breath collection step, the membrane was disassembled and placed inside an adaptor that enabled inserting it into the inlet of the MMTD—the thermal desorption unit (FIG. 9). A typical reading of the MMTD takes about 10 sec. In this experiment, breath was collected from a healthy subject by the apparatus described above. Each sample is the sum of three deep breaths conducted in the lab without any prior air cleaning. After two baseline samples, the subject drank a cup of 100% squeezed orange juice which is known to have high limonene content. The first breath sample was given five minutes after the juice was consumed. The second and third samples were given five minutes after that. Clean Tenax ESM and a membrane with pure limonene standard were also measured as blank and standard. In the graph presented in FIG. 10, it is shown that in all the breath samples taken after the orange juice consumption a significant peak is present which corresponds to the limonene standard measurement.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A fibrous mat comprising a plurality of fibers, wherein each of the fibers is a porous polymeric fiber comprising an aromatic polymer; wherein:
an average cross-section of the fibers is between 0.5 and 30 um;
the aromatic polymer is stable at a temperature of at least 200° C.;
the fibrous mat is characterized by air permeability coefficient of at least 10⁻¹³ m².

2. The fibrous mat of claim 1, wherein the aromatic polymer is capable of binding a volatile organic specie (VOC).

3. The fibrous mat of claim 1, wherein the aromatic polymer comprises poly-2,6-diphenyl-p-phenylene oxide; and wherein said binding is by adsorption; optionally wherein the VOC is a non-polar small molecule.

4. The fibrous mat of claim 3, wherein the VOC comprises an alcohol, a glycol, a terpene, a cyclic compound, an aromatic compound, a chlorinated aromatic compound, a nitro-aromatic compound, an aldehyde, a ketone, an amine, or an amide, including any combination thereof.

5. The fibrous mat of claim 1, wherein said fibrous mat is characterized by at least one of: (i) a density of between 0.05 and 0.1 gr/cm³; (ii) a porosity of between 70 and 90%; (iii) a BET surface area of at least 25 m²/g; and an average pore size of between 1 and 500 nm.

6. The fibrous mat of claim 1, wherein said fibrous mat is characterized by at least one of: (i) an average pore size of between 5 and 50 um; (ii) elongation to break of between 5 and 10%; (iii) Young's modulus between 5 and 10 MPa; and (iv) thermal stability at a temperature up to 500° C.

7. The fibrous mat of claim 2, wherein the fibrous mat is in a form of a self-supporting membrane characterized by a thickness of between 50 and 2000 um; and wherein the self-supporting membrane is configured for sampling said VOC.

8. The fibrous mat of claim 7, wherein the self-supporting membrane is configured to support gas flow at a flow velocity of at most 5 m/sec.

9. The fibrous mat of claim 7, wherein the self-supporting membrane is in a form of a non-woven uniform layer; and wherein the self-supporting membrane is characterized by at least one of: (i) a tensile strength of at least 0.4 MPa; (ii) air permeability coefficient of between 10⁻¹² and 10⁻¹⁴ m².

10. The fibrous mat of claim 7, wherein the self-supporting membrane is capable of adsorbing the VOC so as to obtain a w/w ratio between adsorbed VOC and the self-supporting membrane between 1:1.000.000 and 1:10.

11. The fibrous mat of claim 1, wherein the fibrous mat is characterized by at least one of: (i) a DSC pattern exhibiting at least one endothermic peak in the range of from 470° C. to 500° C.; (ii) a Tg of the aromatic polymer within the fibrous mat is about 223° C.; and (iii) a crystallinity degree of the aromatic polymer within the fibrous mat is 20-50%.

12. A method for detecting a VOC of interest within a sample, the method comprising:
contacting the fibrous mat of claim 1 with a fluid sample comprising a plurality of VOCs under appropriate conditions, thereby obtaining one or more VOCs adsorbed to said fibrous mat;
exposing the one or more VOCs adsorbed to said fibrous mat to a detector configured for detecting said analyte of interest, thereby determining the presence of said VOC of interest within said sample.

13. The method of claim 12, wherein said appropriate conditions comprise at least one of: (i) exposing the fibrous mat to a flowing gaseous sample at a flow rate of at least 0.05 m/sec; (ii) time period sufficient for adsorbing an effective amount of said VOC of interest to said fibrous mat; optionally wherein the effective amount is sufficient for detection of said VOC of interest by said detector.

14. The method of claim 12, wherein a concentration of said VOC of interest within the sample is at least 0.1 ppb.

15. A method for manufacturing the fibrous mat of claim 1, comprising:
forming a polymeric solution comprising the aromatic polymer and a solvent; and
introducing the polymeric solution into an electrospinning apparatus under appropriate conditions, thereby obtaining the fibrous mat.

16. The method of claim 15, wherein the solvent is compatible with electrospinning process; wherein the aromatic polymer has a sufficient solubility within the solvent; optionally wherein said solubility is between 1 and 200 g/L at a temperature of up to 40° C.

17. The method of claim 15, wherein the solvent comprises a chlorinated solvent and a polar solvent; optionally wherein a v/v ratio between the chlorinated solvent and the polar solvent within the polymeric solution is between 7:3 and 9.5:0.5.

18. The method of claim 17, wherein said chlorinated solvent comprises chloroform, DCM, dichloroethane, or any combination thereof; and wherein said polar solvent comprises DMF, DMSO, NMP, methanol, ethanol, or any combination thereof.

19. The method of claim 17, wherein said forming the polymeric solution comprises (i) dissolving a sufficient amount of the aromatic polymer within the chlorinated solvent to obtain a solution, and (ii) subsequently adding a sufficient amount of the polar solvent to the solution, thereby forming the polymeric solution.

20. The method of claim 15, wherein said method further comprises exposing said fibrous mat to a temperature between 100 and 300° C.
